(19) United States
(12) Patent Application Publication
(10) Pub. No.: US 2024/0416141 A1
(43) Pub. Date: Dec. 19, 2024

(54) PHOTOTHERAPY MASK

(71) Applicant: AESTHETIC TECHNOLOGY LIMITED, Warrington, Cheshire (GB)

(72) Inventors: Dale NEEDHAM, Warrington, Cheshire (GB); Huw ANTHONY, Warrington, Cheshire (GB); Isobel REEVES, Warrington, Cheshire (GB)

(73) Assignee: AESTHETIC TECHNOLOGY LIMITED, Warrington, Cheshire (GB)

(21) Appl. No.: 18/687,746

(22) PCT Filed: Sep. 2, 2022
(86) PCT No.: PCT/GB2022/052245

§ 371 Date: Feb. 28, 2024

Related U.S. Application Data
(60) Provisional application No. 63/260,867, filed on Sep. 2, 2021.

(51) Int. Cl. A61N 5/06

(52) U.S. Cl. CPC A61N 5/0616; A61N 2005/0647; A61N 2005/0652; A61N 2005/0659; A61N 2005/0663

(57) ABSTRACT

A LED phototherapy mask. The mask comprises a multilayer construction formed from at least one flexible material so as to be capable of being manipulated to adopt a desired 3D shape profile to fit the contours of a person's face.

### FIELD OF INVENTION

This concept relates to phototherapy device configurable to be worn and cover the face of a person, and in particular, although not exclusively, to a medical and/or cosmetic irradiation mask for delivering phototherapy to an individual's face.

### BACKGROUND

Phototherapy, being ‘treatment with light’ is the application of low-level light energy to stimulate or regulate biological processes with proven therapeutic effects. The effective mechanism is a natural response similar to that of plant photosynthesis through a process known as photobiomodulation. LED Phototherapy (i.e., phototherapy using light-emitting diodes (LEDs) to deliver the light) is well evidenced for its regenerative and anti-inflammatory benefits without creating trauma making it safe and suitable for all skin types. The treatment involves exposing the skin to low levels of beneficial light energy from the visible and infrared part of the light spectrum. Specific wavelengths interact with biological systems and activate key cell receptors which consequently trigger a transfer of light energy to cellular energy. Skin cells that are energised function better and can renew faster to promote youthful, healthy, and radiant skin. With a shift towards non-invasive treatments, LED Phototherapy offers a progressive alternative to more aggressive procedures which carry a higher risk of adverse response, discomfort, and downtime. LED energy delivered via spectrally pure wavelengths stimulate the skin's own repair mechanism, correct problem skin conditions and help to restore optimum skin function. LED Phototherapy is now recognised as an essential modality for skin care practitioners and is well evidenced for the safe and effective treatment of a wide range of inflammatory and problematic skin conditions, accelerated wound healing and ageing skin indications.

Products exist on the market for providing phototherapy to a user's face. These typically comprise a mask having an LED array disposed or embedded on the interior surface of the mask to irradiate a user's face skin with light from the LEDs. Such masks are typically curved to fit the general shape of the face, but they typically curve or bend in only one direction, i.e., along the midline of the face and sit on the face like a shield. Example phototherapy masks are described in WO2018/009270A1; WO2018/196310A1; WO2019/103301A1; WO2019/200686A1; WO2020/003231A1; W2020/040435A1;

WO2021/085886A1 and WO2022/045850A1.

Accordingly, existing designs often do not fit a user's face appropriately leading to irregular or non-uniform irradiation including in particular overexposure and underexposure at certain areas. Accordingly, there is a need for a phototherapy mask that overcome these and other deficiencies.

### SUMMARY OF THE INVENTION

It is an objective of the present concept to provide a phototherapy mask to closely fit the shape and contours of a person's face so as to provide a uniform light treatment of the face skin. It is a further objective to provide a mask that may be manipulated to create multiple curves or bends in the mask in both a lengthwise and a widthwise direction. It is a yet further objective to provide a phototherapy system for efficient and effective light transmission to the skin and to provide a phototherapy device with extended longevity and operational efficiency relative to existing phototherapy devices.

The objectives are achieved by providing a mechanically flexible phototherapy mask having a multilayer construction and an appropriate material composition. The present mask provides a mounting for an array of LEDs capable of emitting light at a mask inner surface positionable opposite and to cover the skin at the face of a person. The present phototherapy device and system comprises suitable electronic components to enable a user to select multiple treatment modes and to activate and deactivate LED chips according to a desired phototherapy session. In particular, the LEDs of the present system comprise a multiple chip design in which the internal chips are thermally partitioned from one another to increase electronic and operational efficiency.

According to a first aspect of the present concept there is provided a phototherapy mask configurable to be worn and cover a forehead, chin and cheeks of a person comprising: a flexible outer layer; a flexible inner layer positionable opposite the skin of a person; a coupling arrangement physically coupling the outer and inner layers together; an LED assembly having a plurality of LEDs and a flexible LED printed circuit board to mount the LEDs, the LED assembly mounted between and positionally retained by the inner and outer layers; wherein the mask is capable of being bent to adopt a curved shape profile at least in lengthwise direction between a forehead end and chin end of the mask.

Reference within the specification to a ‘mechanically flexible phototherapy mask’ encompasses a mask having individual mask layers that are capable of being bent, curved or folded from a particular plane (e.g., a planar configuration) to adopt a curved profile. The material composition and physical construction of the present mask enables the mask to be easily bent, molded and adjusted by the hands (thumbs and fingers) of a user. Additionally, respective inner layers are preferably positioned intermediate an outer and an inner layer such that the respective inner layers comprise positional/motional freedom being allowed to slide over one another as the mask is bent to adopt the 3D shape profile.

Preferably, the mask comprises a cheek slit extending inwardly from each lateral side of the mask towards a central longitudinal axis of the mask. Preferably, each slit is curved along its length corresponding to a widthwise direction across the mask. Preferably, a width of each slit decreases in a direction from a perimeter of the mask towards the central longitudinal axis. Preferably, the mask comprises at each lateral side, a head strap attachment. The head strap attachment preferably comprises a pair of apertures positioned above and below each cheek slit. The head strap may be introduced into the apertures such that when the head strap is placed under axial tension, the apertures are drawn together as the head strap is tightened. This arrangement ‘closes’ or ‘narrows’ a width of the cheek slits so as to position and hold the mask securely at the face of a person. Optionally the head strap or at least a portion of the head strap is elasticated.

Preferably, the mask comprises a pair of eye openings. Preferably, the mask further comprises a mouth opening and/or a nose opening. Preferably, each cheek slit is positioned in a lengthwise direction of the mask between the eye openings and the mouth opening. Preferably, the mask further comprises eye guards extending from or attachable to the eye openings. Preferably, the eye guards comprise a material different to a material of the one or more layers of the mask.

Optionally, the coupling arrangement may comprise a plurality of male projections extending from one of the inner or outer layers and a plurality of holes provided at the alternate outer or inner layer, the projections capable of being received within the holes to couple the inner and outer layers together. Optionally, the coupling arrangement is positioned at or towards a perimeter of the mask and/or the coupling arrangement is positioned exclusively at or towards a perimeter of the mask. As will be appreciated, the coupling arrangement may comprise any means by which the outer and inner layer may be coupled together to entrap and/or encapsulate the inner layers so as to provide a multilayer composite integral body. Optionally, the coupling arrangement is an adhesive, a chemical or thermal bonding (such as heat bonding) to act between the inner and outer layers.

Preferably, the mask comprises an eye guard projecting from each of the respective eye openings at the inner layer, each eye guard comprising a material that is different to a material of the inner and/or outer layer. Preferably, each eye guard is annular to define a skirt at each eye opening.

Preferably, the mask further comprises a light reflector layer positioned intermediate the LED printed circuit board and the inner layer, the reflector layer comprising a plurality of openings through which the LEDs extend in a direction towards the inner layer.

Preferably, the mask further comprises a light diffuser arrangement to diffuse light emitted from the LEDs and transmitted to the skin at the face. Preferably, the diffuser arrangement comprises a plurality of light diffusers positioned proximate to each LED at the inner layer. Optionally, each light diffuser comprises a conical section extending axially in a direction away from each LED at the inner layer. Preferably, each cylindrical section comprises an open end to at least partially receive an LED such that the LED is positioned adjacent an internal chamber defined by the conical section of the light diffuser.

Optionally, the outer layer and/or the inner layer comprise a silicone material. Optionally, the flexible LED printed circuit board comprises at least one polymer substrate and a conductive layer having metallic tracks coupled to or mounted at the polymer substrate.

Optionally, the reflector layer comprises a polymer material, a white or reflective material.

Optionally, each LED is a multi-chip LED, the chips of each LED configured to emit light of a different wavelength. Optionally, each LED comprises a dual, triple or quad chip design. Preferably, each chip is thermally partitioned from one another via at least one physical partition. Such an arrangement enhances the thermal and operational efficiency of the mask such that inactive chips are not heated by active chips. When non-active chips are subsequently activated, their electronic efficiency is enhanced relative to a pre-heated chip. Preferably, the thermal portioning at each chip is provided at or extends from the printed circuit board (PCB) layer.

Preferably, the present device and system comprises an electronic controller electrically coupled to each LED, the controller configured to control a current supply to each LED to switch each chip of each LED between an active and inactive mode. Preferably, the present system further comprises at least one processor; a user interface; a data storage library; an operational mode library or utility; an electronic diagnostic utility; a data storage device; a battery; an external power port; wired or wireless communication means for data transmission; input and output means including audio/visual input and output components. Preferably, the device further comprises a plurality of resistors mounted at the LED printed circuit board. The mask may comprise a resistor per LED with each resistor being electronically called to each respective LED.

Preferably, a material composition of the mask is configured to allow the mask to be bent from a generally planar shape profile to a contoured 3D shape profile approximately matching a contoured 3D shape profile of a face of a person.

According to a further aspect of the present concept there is provided a method of preparing a phototherapy mask to be worn and cover a face of a person comprising: providing a flexible multilayer body having flexible outer and inner layers and an LED assembly mounted between the inner and outer layers, the multilayer body secured together via a coupling mechanism; bending the multilayer body to create a curved shape profile extending at least in a longitudinal direction between a forehead end and a chin end of the mask.

Preferably, the method comprises bending the multilayer body at a position of the cheek slits such that the bend extends laterally widthwise across the mask between respective inner terminal ends of the cheek slits. Preferably, the method may further comprise creating multiple bends in the mask such that the mask adopts a curved shape profile extending in the lengthwise direction between the forehead and chin ends and in a widthwise direction between the lateral sides of the mask.

According to a further aspect of the present concept there is provided a method of irradiating the skin at the face of a person comprising: providing a flexible multilayer body having a flexible outer and inner layers and an LED assembly mounted between the inner and outer layers, the flexible multilayer body comprising a contoured 3D shape profile; powering the LED using at least one battery; via a user interface, selecting a treatment mode from a plurality of treatment modes; via a controller, determining if the battery has sufficient power remaining to supply power to the LEDs to deliver the treatment mode involving a predetermined LED power demand delivered for a predetermined time period; and the controller either activating the LEDs to deliver the treatment mode or outputting a notification to a user that the selected treatment mode will not be activated based on the determined power at the battery.

According to a further aspect of the present concept there is provided a method of irradiating the skin of the face of a person comprising: providing a flexible multilayer body having a flexible outer and inner layers and an LED assembly mounted between the inner and outer layers, the flexible multilayer body comprising a contoured 3D shape profile;

emitting light from the LEDs; passing the light emitted from the LEDs through at least one light diffuser positioned proximate to, opposite and/or adjacent each LED at the inner layer to diffuse the light as it is transmitted to the skin.

According to a further aspect of the present concept there is provided a phototherapy mask configurable to be worn and cover at least a part of a forehead, chin and cheeks of a person comprising: a flexible multilayer arrangement defining an inner surface to be positioned opposite a person's face and an outer surface; and an LED assembly having a plurality of LEDs and a flexible LED printed circuit board to mount the LEDs, the LED assembly mounted between and positionally retained by the inner and outer layers; wherein the mask is capable of being bent to adopt a curved shape profile at least in lengthwise direction between a forehead end and chin end of the mask. According to a further aspect of the present concept there is provided a method of phototherapy using the phototherapy mask as described herein.

According to a further aspect of the present concept there is provided a phototherapy mask comprising: a flexible main body defining an inner surface to be positioned opposite a person's face and an outer surface; and an LED assembly having a plurality of LEDs and a flexible LED printed circuit board to mount the LEDs, the LED assembly mounted at the main body; wherein the main body and the LED assembly are capable of being bent or curved in at least one of a lengthwise and widthwise direction of the mask.

### BRIEF DESCRIPTION OF DRAWINGS

A specific implementation of the present invention will now be described, by way of example only, and with reference to the accompanying drawings in which:

FIG. 1 is a front view of an LED phototherapy mask having a flexible configuration so as to adopt a curved 3D shape profile according to a specific implementation of the present concept;

FIG. 2 is a rear perspective view of the mask of FIG. 1 with layers removed for illustrative purposes;

FIG. 3 is a further front perspective view of the mask of FIG. 1 manipulated into a 3D shape profile with curves in both lengthwise and widthwise directions;

FIG. 4 is a side perspective view of the mask of FIG. 3;

FIG. 5 is a top end view of the mask of FIG. 4;

FIG. 6 is a rear perspective view of the mask of FIG. 5 in a generally planar, non-curved configuration;

FIG. 7 is a side view of the mask of FIG. 6 having the generally planar configuration;

FIG. 8 is a rear perspective view of an eye guard attachable to the mask of FIGS. 1 to 7;

FIG. 9 is a perspective front view of the eye guard of FIG. 8;

FIG. 10 is a perspective rear view of the mask of FIG. 6 with the eye guards of FIGS. 8 and 9 mounted in position at eye apertures;

FIG. 11 is a rear view of the mask of FIG. 10 provided with a head strap to secure the mask to a head of a person;

FIG. 12 is a further rear view of the mask of FIG. 11 with an inner layer removed for illustrative purposes;

FIG. 13 is a rear view of an LED PCB layer of the mask of FIG. 12;

FIG. 14 is a rear perspective view of the mask of FIG. 12 manipulated into a 3D curved shape profile;

FIG. 15 is a cross-section through multiple layers of the mask of FIG. 1 towards a perimeter region of the mask;

FIG. 16 is a further cross-sectional view through the multiple layers of the mask of FIG. 1 towards a central region of the mask;

FIG. 17 is a schematic illustration of various electronic components of the present concept related to the operation of the mask as described herein.

### DETAILED DESCRIPTION OF PREFERRED EMBODIMENT OF THE INVENTION

The present concept relates to methods and systems (specifically masks) for delivering LED phototherapy to a user's face. Embodiments of the methods are a non-invasive treatments that deliver clinically proven therapeutic light energy for rejuvenating and corrective benefits. Specific wavelengths are absorbed by the skin to stimulate cell renewal and collagen production, resolve problem skin conditions such as acne and redness and accelerate skin healing. It is a safe and pain-free treatment without downtime. Users can expect immediate improvement in skin tone, hydration and luminosity whilst the beneficial light simultaneously stimulates deeper cell processes for longer lasting benefits. For sensitive skin conditions, the present methods offer relief without redness and irritation. Phototherapy can be used for the treatment of acne, psoriasis, sensitive skin, musculoskeletal pain and other skin conditions and can also provide wound healing. More cosmetic treatments include skin rejuvenation, reduced pigmentation and redness.

LED energy delivered via spectrally pure wavelengths stimulate the skin's own repair mechanism, correct problem skin conditions and help to restore optimum skin function. Successful treatment with LED Phototherapy is determined by delivering clinically proven wavelengths at an optimised intensity to maximise the light/chromophore interaction that allows these specific cellular reactions to take place. A chromophore is a target compound within the skin having unique light absorbing properties. If the wavelength does not match the target chromophore there will be no absorption, no reaction and no result. Also, the desired reaction may not occur if the delivered optical power is too low.

Embodiments of the disclosed methods and systems are configurable to deliver blue 415 nm, red 633 nm, near infrared 830 nm which are the industry leading and most clinically evidenced wavelengths for the LED Phototherapy treatment. The present concept is also configurable for operation to emit other wavelengths as desired. Each wavelength of light is absorbed by a different target chromophore to stimulate specific skin enhancing processes with proven therapeutic effects. The disclosed methods and systems enable these beneficial wavelengths to be delivered in single or multi-wavelength mode via a plurality of protocol (or mode) options, offering a tailored and adaptable treatment approach. Multi-wavelength treatments target a range of indications in one session for maximum skin enhancing results.

Blue Light 415 nm (penetration up to 1 mm): KEY MECHANISM: ANTI-BACTERIAL. Blemish fighting blue light destroys the bacteria which causes acne and helps in the prevention of breakouts. Blue light is absorbed by p. acnes bacteria and triggers a natural photochemical reaction releasing singlet oxygen. Singlet oxygen has a powerful antibacterial action to help eliminate spots whilst being gentle on the skin. It also helps to balance oil production and improve skin clarity. Applied in combination with red and near infrared light, the present treatment offers enhanced results for acne and is excellent as a post treatment procedure to help minimise breakouts and reduce redness.

Red Light 633 nm (penetration 2 to 3 mm): KEY MECHANISM: REJUVENATION AND CELL RENEWAL. Rejuvenating red light accelerates cell renewal and repair, boosting collagen and elastin synthesis for smoother firmer skin. Red light is absorbed in the cell mitochondria and stimulates the synthesis of Adenosine Triphosphate (ATP), an essential energy for cellular function. Supercharging our cells triggers a cascade of beneficial biological reactions which result in a range skin enhancing effects. Skin cells that are energised function better can regenerate up to 200% faster. Red light is potently absorbed by fibroblasts increasing collagen and elastin synthesis and boosting hydration levels. Improved blood flow brings increased tissue oxygenation to accelerate repair whilst stimulation of the lymphatic systems helps with detoxification. Red light offers immediate improvement in skin tone, hydration and vitality whilst the beneficial light simultaneously stimulates cellular processes for long lasting benefits in appearance and health of the skin.

Near Infrared Light (NIR) 830 nm (penetration 5 to 10 mm): KEY MECHANISM:
WOUND HEALING ACTION. Near Infrared light is absorbed in the skins deepest layers and works synergistically with Red light for optimum rejuvenation results. Exposure to near infrared light at 830 nm increases blood circulation bringing vital oxygen and nutrients to help modulate inflammation, accelerate wound healing, calm irritation and reduce redness. Near infrared light builds strength and integrity for compromised, environmentally damaged and sensitive skin conditions. It is also clinically effective in the treatment of hyperpigmentation.

Embodiments of the present apparatus, system and method may comprise singular, twin, triple or other multiple chip LEDs to deliver the targeted wavelengths required to enable the treatment of one or more conditions such as acne, psoriasis, wound healing and muscular skeletal pain etc. Indications for use include but are not limited to the following conditions Skin Rejuvenation; Complexion; Dry Skin; Pigmentation: Photo-damage; Pigmentation; Acne: Mild to Moderate; Acne: Moderate to Severe; Redness: Vascular; Redness: Skin Tone; Sensitive Skin; Skin: Problem conditions; Psoriasis; Wound Healing;

Pain. LED phototherapy works by delivering consistent volumes of light across the targeted area of the user. Due to inverse square law, the volume of optical power can vary based on the distance in which and LED is situated from the users target area (skin). Existing masks sit on the user's face as a shield, such that the LED's on the lower half of the face and across the forehead are situated at a greater distance from the target area than the LED's around the cheeks and nose. Consequently, a user receives varying levels of optical power across the target area, which leads ultimately to an inconsistent treatment due to the differential optical power delivered.

The present concept includes embodiments having a 2D-3D flexible design that enables the user to manipulate the mask into a multiple-curved 3D form. This enables an operator or user to mould the mask to sit evenly across the face such that the array or LEDs, distributed over an inner surface the mark, are positioned at a generally uniform separation distance from the skin at all regions of the face. This enables the device to deliver a more uniform and consistent treatment via the optical power delivered from the LED's. To improve the fit, an elasticated feature on the head strap encourages the masks to fit and be maintained in position across the face, opposite the skin, to ensure consistent light irradiation throughout the duration of the treatment.

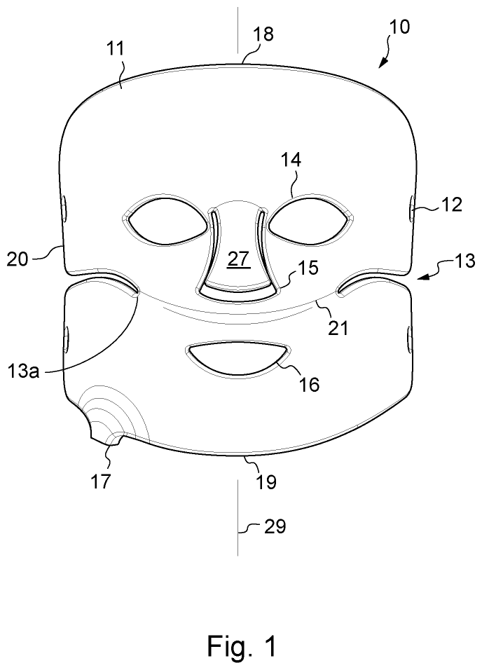

Referring to FIG. 1, the LED phototherapy mask 10 comprises an outward facing or external surface 11 being defined at a perimeter by a first lengthwise edge (or end) 18 positionable at the forehead of a person; a second lengthwise edge (or end) 19 positionable at a chin region of a person; and two lateral edges (or sides) 20 extended lengthwise between ends 18, 19. A plurality of strap apertures 12 extend through the mask 10 towards a perimeter of the mask at each lengthwise side 20. Mask 10 further comprises a pair of eye openings 14, a nose slit 15 and a mouth opening 16. A nose guard 27 extends between eye openings 14 towards mouth opening 16 at the region of slit 15 so as to extend over a majority of the nose of the person when mask 10 is mounted at the face. Mask 10 is symmetrical about longitudinal axis 29 save for an electronic coupling 17 (e.g., a micro-USB port) provided at or towards mask chin end 19. A pair of slits extend widthwise across mask 10 from each lateral side 20. Each slit 13 is positioned in a lengthwise direction between eye openings 14 and mouth opening 16 so as to be positioned at approximately at a lower end of region of nose slit 15. Slits 13 therefore correspond to a position approximately at the cheeks of a person wearing mask 10.

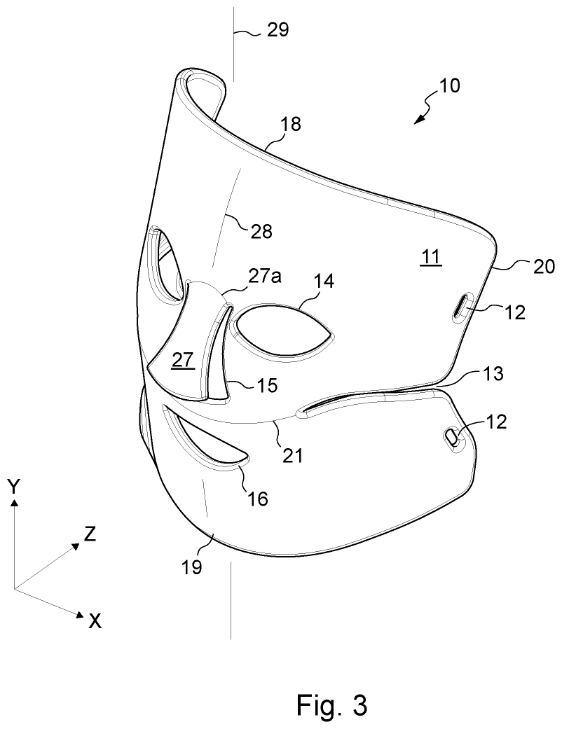

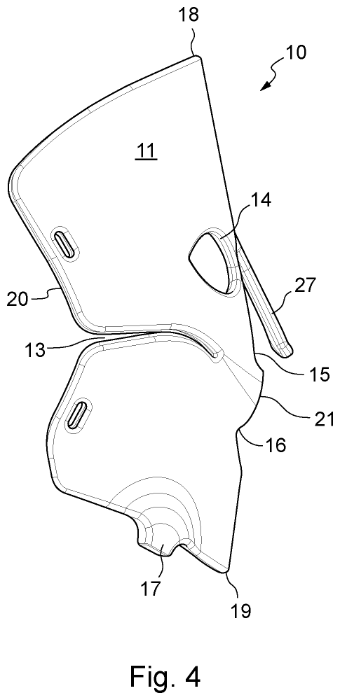

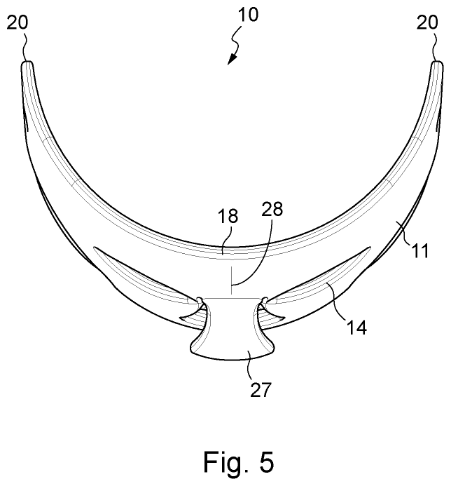

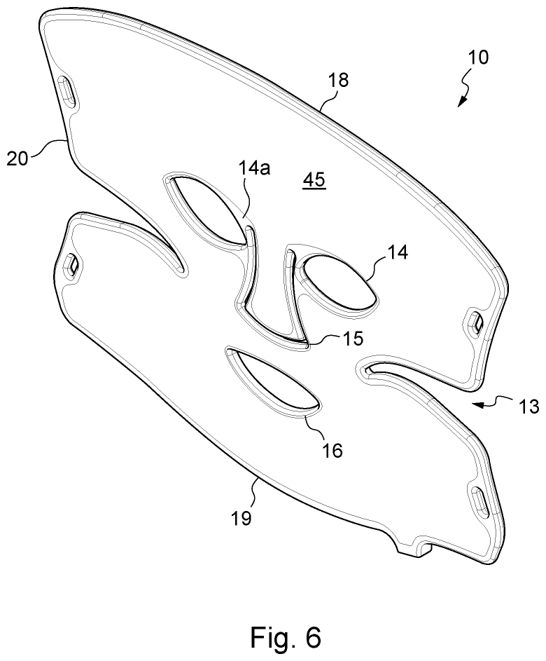

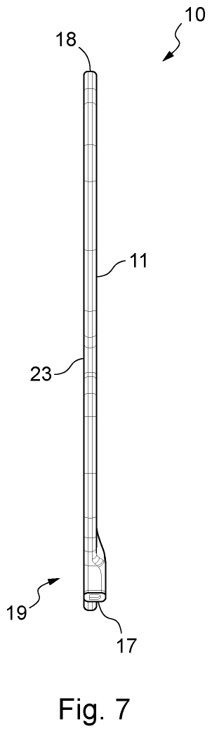

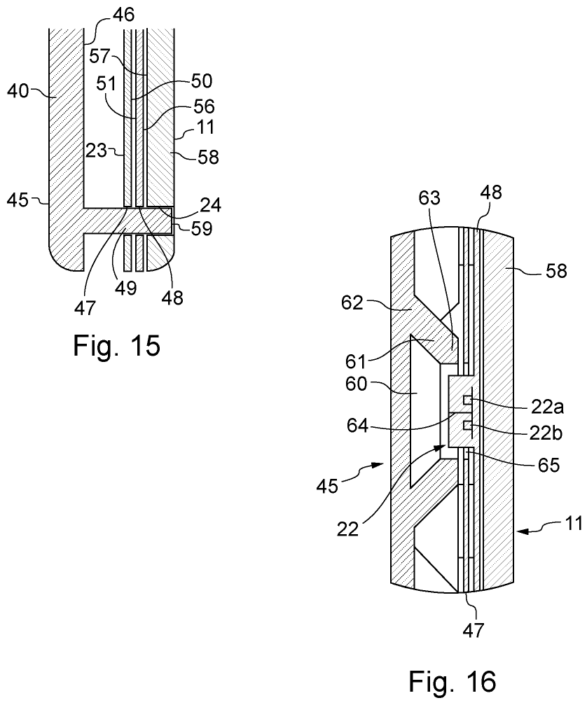

The material composition of mask 10 is such that the mask is designed to be flexible and to be easily bent, curved and moulded by the hands and fingers of an operator so as to be capable of being adapted in a 3D configuration to correspond to the general curved shape profile of a person's face. Such curvature includes a first generally bend or curve (28) extending in the lengthwise direction and a second bend or curve 21 extending in a widthwise direction across the mask. Cheek slits 13 facilitate a user bending mask 10 in that the bend 21 extends between the innermost lengthwise ends 13a of the slits 13. FIGS. 3-5 further illustrate the manner in which mask 10 may be curved in both lengthwise and widthwise directions. In particular, bend or curve 28 extends in the lengthwise direction i.e., the same direction as a longitudinal axis 29 of the mask 10. Referring specifically to FIG. 3, curve 28 extends generally in the z direction whilst curve 21 extends generally in the x direction. As such mask 10 is bent into the z direction via the first and second curves 28, 21. Additionally, due to the material composition of mask 10, nose guard 27 is also adjustable and may be bent or hinged to open and close slit 15 via a pivoting about nose bridge 27a extending laterally between eye holes 14. FIG. 4 illustrates how the forehead end 18 and chin end 19 are bent rearwardly rearwards (in the z direction) away from a forwardmost part of mask 10 (corresponding to nose guard 27) via the widthwise bend 21. Similarly, FIG. 5 illustrates how lateral sides 20 are bent rearwardly (in the z direction) from nose guard 27 via the lengthwise bend 28. FIGS. 6 and 7 illustrate the flexible material configuration of mask 10 to be capable of being manipulated between a 2D generally flat or planar shape profile and a 3D fully contoured shape profile. The present mask 10 is therefore fully adjustable to comprise multiple bends, folds and angled orientations as desired. Such configuration is provided firstly by the mask material composition and secondly via a multi-layered construction as illustrated and described referring to FIGS. 15 and 16.

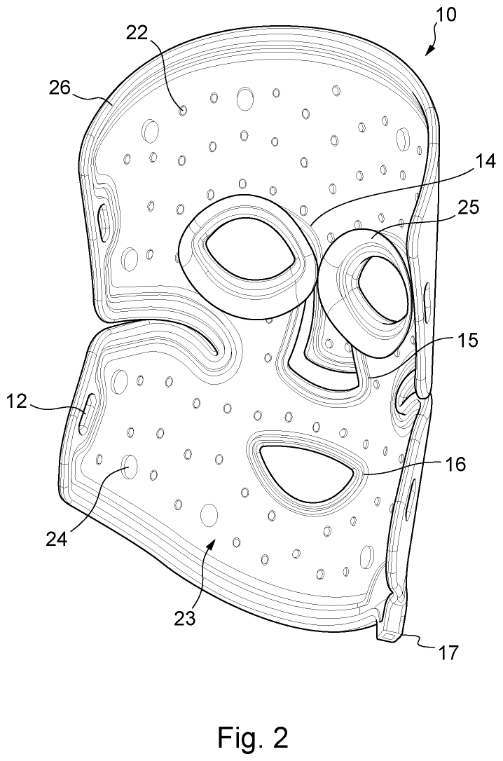

Referring to FIG. 16, mask 10 comprises an inner layer 40 having inward facing surface 45 intended for positioning opposite the skin of a person. Mask 10 also comprises an outer layer 58 being external facing and having outward or external facing surface 11. At or towards a perimeter of mask 10, inner layer 40 is provided with a plurality of projections, bosses or lugs 49. A plurality of corresponding holes 24 are provided at outer layer 58 so as to receive respective end regions 59 of lugs 49. Such a configuration provides a coupling arrangement to physically attach the outer and inner layers 40, 58 as a unitary body. Lug ends 59 and holes 24 may be configured with appropriate friction fitting components (for example bayonet, deformable lugs, detents, washers etc.) so as to provide a click-lock type coupling arrangement for the secure assembly of the multilayer construction. Mask 10 further comprises a plurality of inner layers including in particular a light reflector layer 47 and an LED printed circuit board (PCB) layer 48. Each layer 47, 48 is provided with a plurality of respective apertures so as to enable lugs 49 of inner layer 40 to mate with the outer layer 58. However, due to the relative dimensions of the apertures at layers 47, 48, such layers are provided with a degree of positional freedom when sandwiched between outer and inner layers 40, 58. This allows the inner layers 47, 48 to slide over one another and to further slide and have positional freedom relative to the outer and inner layers 40, 58 as mask 10 is bent in both the lengthwise and widthwise directions to adopt the curved shape profile of FIGS. 2 to 5.

According to the layered construction of FIG. 15, inner layer 40 also comprises internal face 46 positioned opposite the internal face 23 of reflector layer 47. PCB layer 48 comprises internal surface 51 positioned opposite internal surface 50 of reflector layer 47. LED PCB layer further comprises internal surface 56 positioned opposite internal surface 57 of outer layer 58.

Referring to FIG. 16, the inner layer 40 is moulded to provide a unique optical delivery system that enables wide dispersion of light at close to the skin. The unique design aspect of this optic is that it does not outcouple the light and guide the light into a focused area, but instead reflects and refracts a proportion of the light internal to the optic and which results in the light illuminating across a wide area (dispersion) and avoid the creation of dark spots where light has been on the surface of skin and over saturated the cell. In particular, inner layer 40 is provided with a plurality of diffusers 61 projecting from internal surface 46 towards outer layer 58. Each diffuser 61 comprises a generally frustoconical shape to define an internal void or region 60 that is surrounded by the conical wall of diffuser 61 extending between an inner end 62 (at layer 40) and an outer end 63 (at/near PCB layer 48). The annular diffuser outer end 63 is open so as to be positioned and extend around an LED 22 mounted at the PCB layer 48. Accordingly, each LED 22 is positioned adjacent the reduced diameter end of the conical diffuser 61. According to the preferred embodiment, each LED 22 projects at least partially into the void 60 via the open end of each respective diffuser 61.

According to the preferred embodiment, inner layer 40 comprises a medical grade silicone.

Preferably, outer layer 58 is also formed from medical grade silicone and is the same or similar to inner layer 40. Such material may comprise existing and common silicone materials for medical applications. As will be appreciated, such silicones may be manufactured via addition or condensation curing techniques to achieve the required density and physical and mechanical characteristics including in particular softness, flexural strength etc. Reflector layer 47 may comprise a polymer material (such as a polyalkylene, polypropylene, polyethylene etc) or a metallic material such as aluminum or similar and is reflective and non-light transmissive so as to reflect any light from LEDs 22 back towards the skin (positioned underneath inner layer 40). The reflector layer 47 may comprise a white PTFE sheet, for example, and may have windows 65 configured to let an LED 22 project through. PCB layer 48 may comprise a multilayer construction having a polymer substrate that supports a plurality of metallic tracks. The PCB layer 48 may further comprise additional polymer layers to sandwich or at least partially encapsulate the electrically conductive tracks within one or more electrically insulating polymer layers.

Optionally, the polymer substrate and/or layers may comprise a polyimide, a polyalkylene such as polypropylene, polyethylene etc. Such a configuration provides for the desired flexibility characteristic of the PCB layer 48.

Each LED 22, according to the preferred embodiment, comprises a first chip 22a and a second chip 22b having a different light emission wavelength. For example, chip 22a may be configured to emit red light and chip 22b may be configured to emit infrared light. As will be appreciated, mask 10 may be provided with LEDs 22 having any configuration and multiplicity of chips 22a, 22b to emit light of different wavelengths within the desired regions of the electromagnetic spectrum suitable for medical and cosmetic phototherapy.

For example, each LED 22 may comprise a dual chip configuration or may comprise three, four or more chips each delivering at least two different light wavelengths. LEDs 22 are distributed generally uniformly at PCB layer 48, as illustrated in FIG. 2 in which inner layer 40 has been removed for illustrative purposes. Accordingly, when activated, each LED 22 and a particular one of the activated chips 22a, 22b is configured to emit light generally over the entire or at least a majority of the inner surface 45. Via diffusers 61, the emitted light is projected as a diffuse illumination onto the skin and to specifically avoid reduced surface area light concentrations that may harm the skin (as very focused irradiation zones). The present configuration and in particular the multi-layered construction including diffusers 61 provides a generally uniform light glow of the desired intensity onto the regions of the skin of the person. The present twin LED design comprises two inner LED sections or regions that are thermally divided by a thermal partition 64. This encourages better thermal dissipation from the chip and improves optical efficiency. Earlier LED masks typically use a 5050 LED with a 6-pin configuration. The three chips are all mounted inside of the same LED well such that when two or more chips are operational at the same time, the heat generated is dissipated across the LED surface area/mounting to PCB board. This results in optical losses due to the increased operating temperatures and heat dissipation to the neighbouring chips that are inactive. By thermally partitioning/isolating the LED chips 22a, 22b via partition 64, the present mask positionally and thermally isolates the chips from one another giving the product an optical efficiency increase. The increase thermal stability results in more optical power per LED and improves lifetime performance.

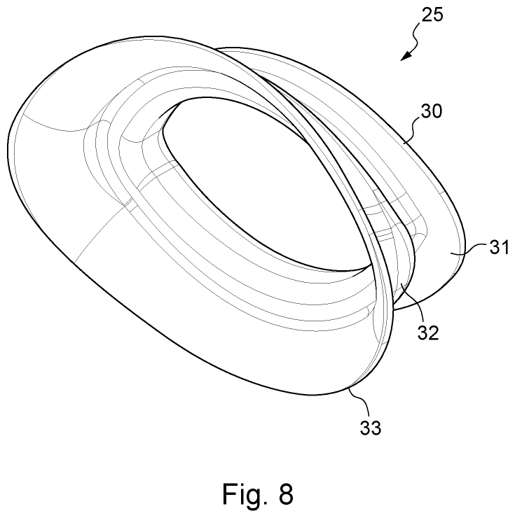

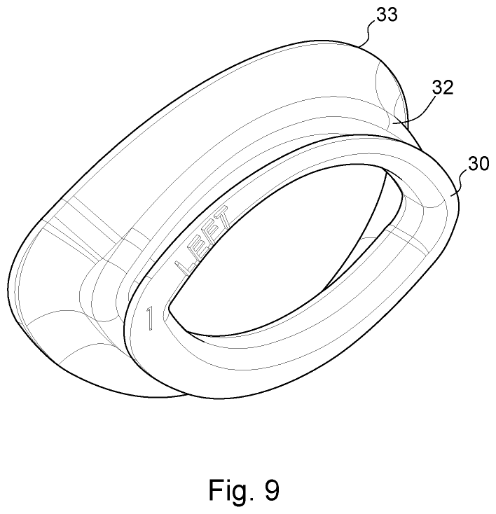

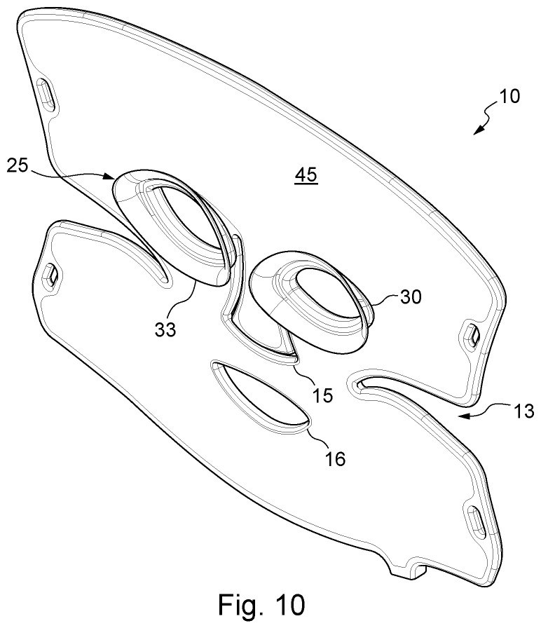

Referring to FIGS. 6, 8, 9 and 10, mask 10 is provided with a pair of eye guards 25. Guards 25 comprise a different material and/or composition to that of layers 40, 47, 48 and 58. Inner layer 40 is light transmissive whilst eye guards 25 comprise of a non-light transmissive material. Each guard 25 comprises a generally annular shape profile. In particular, each guard 25 comprises a first annular lengthwise end 30 having a dimension and shape profile to be positioned and to extend around a respective eye opening 14. Each eye opening 14 is provided with an annular groove 14a (optionally having a lip, shoulder or stepped annular region) to allow eye guard 25 to be clipped into mating contact at each eye opening 14. Each guard 25 further comprises an annular collar 31 positioned adjacent first end 30, an annular skirt 32 extending from collar 31 with skirt 32 terminating at annular guard second end 33. End 33 is radially enlarged relative to first end 30 and comprises a generally oval shape profile in the circumferential direction so as to match the contours and general shape of a person's eye socket. Accordingly, with guards 25 secured in position at eye openings 14, annular ends 33 are be provided in close contact with the skin at each eye socket so as to fully spatially partition and isolate each eye from the irradiation emitted by the LEDs 22. The eye shields 25 protect the eyes from off angle light distribution. Medical devices are subject to rigours photonic testing for both the user and the practitioner where applicable. Typically such devices state their risk classification for both blue and NIR light. Due to the proximity of the mask, the light is deemed to be a higher risk classification which then requires protective eyewear. The eye shields 25 follow the principles of swimming goggles and are designed to suit multiple profiles of faces and nest comfortably around the eye at the eye socket. The material composition is dense, to reduce and preferably block completely the overall light exposure to the eye when in use. Preferably the material of guards 25 is non-light transmissive to block completely the irradiating light. Preferably the material of guards 25 is at least partially flexible for a compression fitting at the eye socket.

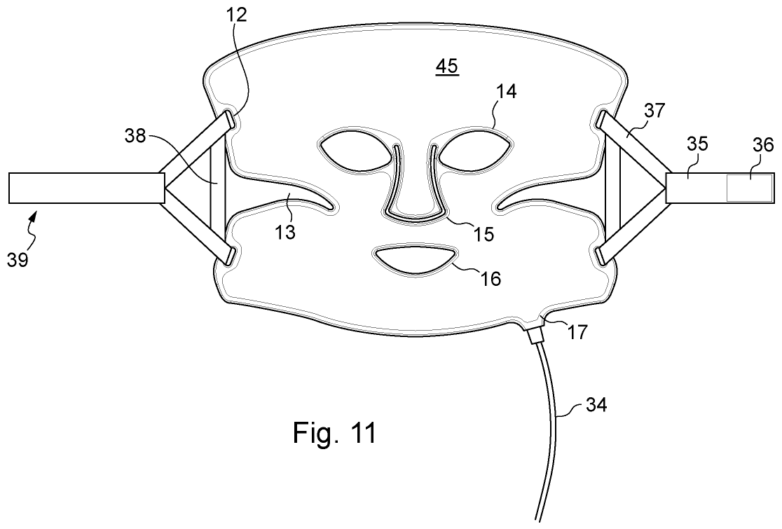

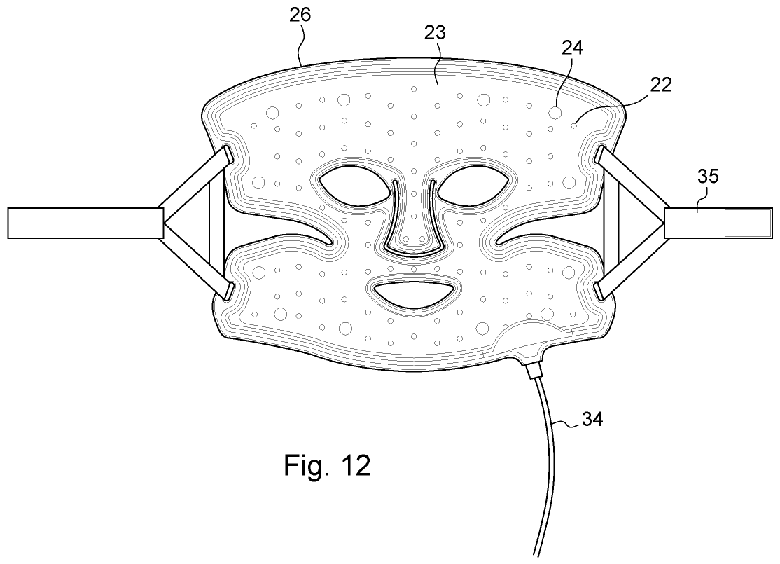

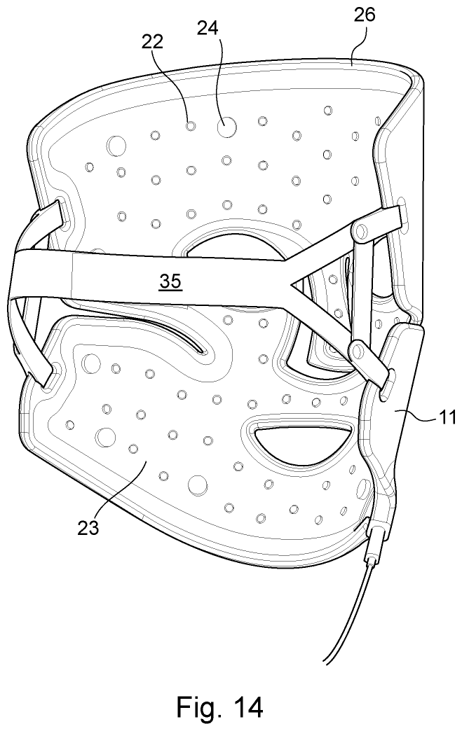

Referring to FIGS. 11 and 12, mask 10 is provided with a head strap 35 so as to secure mask 10 to the head of a person. Strap 35 according to the preferred embodiment is divided into two sections each secured to a respective lateral side via strap apertures 12. In particular, a strap longitudinal section at a first end comprises a respective hook and loop fastening 36, 39 and at a second end comprises a pair of brace straps 37 and cross strap 38 so as to provide a generally triangular attachment region from which the longitudinal strap region extends. As noted, the split-cheek design includes slits 13 at the cheek regions. The cross strap 38 is elasticated between the upper and lower fixing points to automatically close cheek slits 13. Hook and loop fastenings may then be secured at the back of a person's head to hold the mask at a person's face as illustrated in FIG. 14.

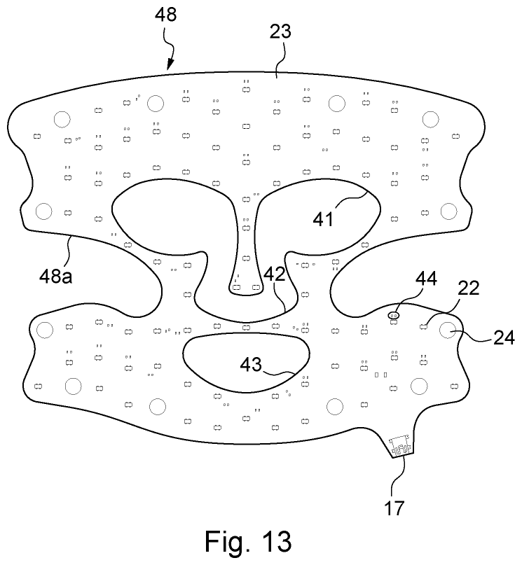

Referring to FIG. 13, PCB layer 48 further comprises a plurality of resistors 44 associated with each respective LED 22 configured to control current to the LEDs 22. Each resistor 44 forms part of the electronic construction of the mask to provide the desired functionality and control of the irradiation according to predefined treatment requirements and modes. PCB layer 48 also comprises corresponding eye, nose and mouth openings 41, 42 and 43 together with cheek slits 48a to correspond to the general shape and configuration of mask 10 as illustrated in FIGS. 1-6.

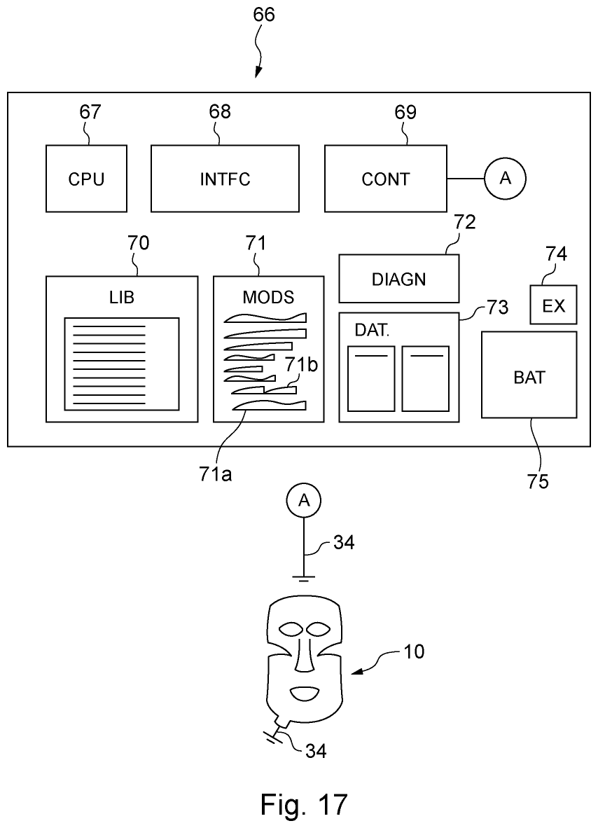

Referring to FIG. 17, the present concept provides an LED based phototherapy mask and irradiation system having an electronic architecture 66 for the activation and control of the irradiation emitted from LEDs 22. In particular, the electronic architecture 66 may be provided at mask 10 locally relative to the multiple layers 40, 47, 28, 58. Alternatively, or in addition, selected components are preferably housed within a suitable control unit positioned remote from mask 10 and attachable via suitable electrical connections 34, in turn attachable to electronic coupling 17 at mask 10 which is mounted at PCB layer 48. The electronic control unit comprises the central processing unit (CPU) 67 (typically one of a plurality of processors) and a user interface 68. The interface 68 may comprise a screen (such as a touch-screen), buttons, dials and the like suitable for control of the activation and power output of the LEDs. A control module 69 is provided with appropriate software, control units, components and functionality so as to implement different operational modes having pre-defined LED operating time/duration and power output level. The present system also comprises at least one library 70 (optionally a data reference library, user data library or other data collection; selectable operation modes (implemented as software); a diagnostic utility 72 (implemented as software); a data storage utility 73; a battery 75; and optionally an external power port 74 for connection to an external power supply. Modes utility 71 comprises a plurality of pre-set and/or configurable modes of operation for the LEDs 22. Such modes include control of the time duration of illumination and a power output of the LEDs. Optionally, a first mode 71a may be configured with a pre-set phototherapy treatment time of 20 minutes in which the LED power is increased steadily from a start point to rise to a maximum and then to decrease towards an end point. An example second mode 71b may comprise a staged interval-based treatment in which the LEDs are powered for a pre-set time, are deactivated and then reactivated for further time intervals. Diagnostic utility 72 may be implemented to run continuously whilst power is supplied to the LEDs to monitor electronic performance and function and to provide notification via interface 68 of any operational errors or problems.

The electronic architecture 66 may further comprise additional electronic components to provide wired or wireless communication with local networks, cloud storage or the internet. The present system may also comprise sensors including for example a light sensor, a temperature sensor, a proximity sensor, a pressure sensor, a voltmeter, ammeter and/or a timer. The various electronic components of architecture 66 as illustrated in FIG. 17 and including LEDs 22 are interconnected and operational according to conventional systems as will be appreciated. Optionally, mask 10 may be coupled electronically to the remainder of the electronic architecture 66 of FIG. 17 via wireless communication. Optionally, battery 75 may be provided locally at mask 10 with the battery controllable wirelessly to activate or deactivate the LEDs 22 according to the mode utility 71, as controlled by control unit 69. In operation, having adjusted the mask to the desired 3D shape profile and secured the mask in position at the face, a user/operator May select a mode (e.g., 71a), from modes utility 71 via interface 68 and/or control unit 69 for a predetermined phototherapy session.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A phototherapy mask configurable to be worn and cover a forehead, chin and cheeks of a person comprising:
a flexible outer layer;
a flexible inner layer positionable opposite the skin of a person;
a coupling mechanism physically coupling the outer and inner layers together;
an LED assembly having a plurality of LEDs and a flexible LED printed circuit board to mount the LEDs, the LED assembly mounted between and positionally retained by the inner and outer layers;
wherein the mask is capable of being bent to adopt a curved shape profile at least in lengthwise direction between a forehead end and chin end of the mask.

2. The mask as claimed in claim 1 comprising a cheek slit extending inwardly from each lateral side of the mask towards a central longitudinal axis of the mask.

3. The mask as claimed in claim 2 wherein each slit is curved along its length corresponding to a widthwise direction across the mask.

4. The mask as claimed in claim 2, wherein a width of each slit decreases in a direction from a perimeter of the mask towards the central longitudinal axis.

5. The mask as claimed in claim 1, comprising a pair of eye openings.

6. The mask as claimed in claim 5 comprising a mouth opening and/or a nose opening.

7. The mask as claimed in claim 2, wherein each cheek slit is positioned in a lengthwise direction of the mask between the eye openings and the mouth opening.

8. The mask as claimed in claim 1, wherein the coupling mechanism comprises a plurality of male projections extending from one of the inner or outer layers and a plurality of holes provided at the alternate outer or inner layer, the projections capable of being received within the holes to couple the inner and outer layers together.

9. The mask as claimed in claim 8 wherein the coupling mechanism is positioned at or towards a perimeter of the mask and/or the coupling mechanism is positioned exclusively at or towards a perimeter of the mask.

10. The mask as claimed in claim 5, further comprising an eye guard projecting from each of the respective eye openings at the inner layer, each eye guard comprising a material that is different to a material of the inner and/or outer layer.

11. The mask as claimed in claim 10 wherein each eye guard is annular to define a skirt at each eye opening.

12. The mask as claimed in claim 1, further comprising a light reflector layer positioned intermediate the LED printed circuit board and the inner layer, the reflector layer comprising a plurality of openings through which the LEDs extend in a direction towards the inner layer.

13. The mask as claimed in claim 1, further comprising a plurality of light diffusers positioned proximate to each LED at the inner layer.

14. The mask as claimed in claim 13 wherein each light diffuser comprises a conical section extending axially in a direction away from each LED at the inner layer.

15. The mask as claimed in claim 1, wherein the outer layer and/or the inner layer comprise a silicone material.

16. The mask as claimed in claim 1, wherein the flexible LED printed circuit board comprises at least one polymer substrate and a conductive layer having metallic tracks coupled to or mounted at the polymer substrate.

17. The mask as claimed in claim 12, wherein the reflector layer comprises a polymer material, a white or reflective material.

18. The mask as claimed in claim 1, wherein each LED is a multi-chip LED, the chips of each LED configured to emit light of a different wavelength.

19. The mask as claimed in claim 18 further comprising thermal partitioning to thermally partition the chips at each LED.

20. The mask as claimed in claim 19 further comprising an electronic controller electrically coupled to each LED, the controller configured to control a current supply to each LED to switch each chip of each LED between an active and inactive mode.

21. The mask as claimed in claim 1, wherein a material composition of the mask is configured to allow the mask to be bent from a generally planar shape profile to a contoured 3D shape profile approximately matching a contoured 3D shape profile of a face of a person.

22. The mask as claimed in claim 1, further comprising a plurality of resistors mounted at the LED printed circuit board.

23. A method of preparing a phototherapy mask to be worn and cover a face of a person comprising:
providing a flexible multilayer body having flexible outer and inner layers and an LED assembly mounted between the inner and outer layers, the multilayer body secured together via a coupling mechanism;
bending the multilayer body to create a curved shape profile extending at least in a longitudinal direction between a forehead end and a chin end of the mask.

24. The method as claimed in claim 23 wherein the mask comprises a cheek slit extending inwardly from each lateral side of the mask towards a central longitudinal axis of the mask.

25. The method as claimed in claim 24 comprising bending the multilayer body at a position of the slits such that bend extends laterally widthwise across the mask between respective inner terminal ends of the cheek slits.

26. The method as claimed in claim 23, comprising creating multiple bends in the mask such that the mask adopts a curved shape profile extending in the lengthwise direction between the forehead and chin ends and in a widthwise direction between the lateral sides of the mask.

27. A method of irradiating the skin at the face of a person comprising:
providing a flexible multilayer body having a flexible outer and inner layers and an LED assembly mounted between the inner and outer layers, the flexible multilayer body comprising a contoured 3D shape profile;
powering the LED using at least one battery;
via a user interface, selecting a treatment mode from a plurality of treatment modes;
via a controller, determining if the battery has sufficient power remaining to supply power to the LEDs to deliver the treatment mode involving a predetermined LED power demand delivered for a predetermined time period; and
the controller either activating the LEDs to deliver the treatment mode or outputting a notification to a user that the selected treatment mode will not be activated based on the determined power at the battery.

28. A method of irradiating the skin of the face of a person comprising:
providing a flexible multilayer body having a flexible outer and inner layers and an LED assembly mounted between the inner and outer layers, the flexible multilayer body comprising a contoured 3D shape profile;
emitting light from the LEDs;
passing the light emitted from the LEDs through at least one light diffuser positioned proximate to, opposite and/or adjacent each LED at the inner layer to diffuse the light as it is transmitted to the skin.
